(19) United States
(12) Patent Application Publication
(10) Pub. No.: US 2011/0311626 A1
(43) Pub. Date: Dec. 22, 2011

(54) CONTROLLED RELEASE COMPOSITIONS COMPRISING ANTI-CHOLINERGIC DRUGS

(76) Inventors: Gopi Venkatesh, Vandalia, OH (US); Vincent Parrino, Doylestown, PA (US); Paolo Gatti, Sesto San Giovanni (IT); Flavio Fabiani, Ronco Briantino (MI) (IT)

(21) Appl. No.: 13/202,957

(22) PCT Filed: Feb. 23, 2010
(86) PCT No.: PCT/US10/25083

§ 371 Date: Aug. 23, 2011

Related U.S. Application Data
(60) Provisional application No. 61/154,504, filed on Feb. 23, 2009.

(51) Int. Cl. A61K 9/22; A61K 31/215; A61P 1/00; A61P 25/04; A61P 25/08; A61P 3/10; A61P 31/00; A61P 25/16; A61P 29/00; A61P 9/00; A61P 25/00; A61P 1/08; A61P 25/18; A61P 11/06; A61P 21/02; A61K 9/14

(52) U.S. Cl. 424/468; 424/495; 514/529; 424/494; 424/501

(57) ABSTRACT

The present invention provides compositions comprising dicyclomine, or salts, and/or solvates and methods of making and using the compositions to treat intestinal hypermotility or Irritable Bowel Syndrome (IBS). The present invention also provides once-a-day orally disintegrating dosage forms comprising compositions of the present invention.

### CROSS REFERENCE TO RELATED APPLICATIONS

The present application claims priority to U.S. Provisional Application No. 61/154,504 filed Feb. 23, 2009, which is incorporated herein by reference in its entirety for all purposes.

### FIELD OF THE INVENTION

This invention relates to compositions comprising anti-cholinergic drugs such as dicyclomine, and methods of making and using such compositions.

### BACKGROUND OF THE INVENTION

Anti-cholinergic drugs block the activity of acetylcholine on cholinergic receptors on the surface of smooth muscle cells, and as a class have utility for a variety of clinical applications, e.g., as smooth muscle relaxants, antispasmodics, anti-motion sickness agents, antihistamines, bronchodilators, etc.

Anti-cholinergic drugs can be difficult to formulate into controlled release dosage foams which provide therapeutic levels of the drug over a 24 hour period using a once-a-day dosing schedule, while minimizing dose related side affects. The difficulty in designing extended release once-a-day dosage forms can be further complicated by the pharmacokinetic properties and physical properties of the drug itself. For example, bicyclohexyl-1-carboxylic acid, 2-diethylamino ethyl ester (dicyclomine, also known as dicycloverine) is an exemplary anti-cholinergic drug known to have smooth muscle relaxant properties. The currently marketed formulation of dicyclomine has a very rapid dissolution profile that results in a rapid rise in blood plasma concentrations of the drug shortly after administration (Tₘₐₓ of approximately 1.5-3 hours) and is eliminated quickly with a short half-life (t_{1/2}) of 1.8 hours. The combination of this rapid Tₘₐₓ and short half life requires that conventional dicyclomine dosage forms be administered multiple times a day in order to maintain, tolerable, therapeutic serum levels over a 24 hour period.

Anti-cholinergic drugs such as dicyclomine HCl are indicated for managing abdominal spasms and pain associated with moderate-to-severe irritable bowel syndrome. Dicyclomine, a muscarinic M1 acetylcholine receptor antagonist, acts as a smooth muscle relaxant and is used as an antispasmodic to alleviate abdominal pain and bloating caused by colonic spasms associated with irritable bowel syndrome (IBS). IBS may be attributed to autonomic neuropathy; decreased vagal tone will lead to constipation while an increase in sympathetic stimulus is associated with diarrhea. The majority of IBS cases are a result of the interrelation between psychological morbidity and visceral hypersensitivity. IBS patients have higher incidences of anxiety, depression and sleep disturbance. Typical anti-cholinergic side effects, such as dry mouth, dizziness, blurred vision and nausea, are problematic for moderate-to-severe IBS patients taking an immediate release therapeutic agent several times a day on an extended basis. Severe symptoms are frequent, intense, and chronically interfere with daily functioning. Moderate-to-severe symptoms also impact social well-being, as patients will tend to avoid long journeys or going out (Drossman, D. 2006 Gastroenterology 20 (5): 121-132 and Smith, D. G. 2005 Am J Manag Care 11: S43-S50). According to the Bentyl® (immediate release dicyclomine HCl capsules) package insert, 46 out of 100 patients in a clinical trial could not take the recommended 160 mg daily dose due to side effects. The prevalence rate of IBS in the U.S. is 15-20% of the general population. As such, conventional dosage forms of dicyclomine are far from clinically optimal, not only for patient compliance reasons, but also because a rapid serum level rise to Cₘₐₓ is associated with common side effects such as dry mouth, dizziness, blurred vision, nausea, etc.

Thus, there is a need for controlled release formulations of anti-cholinergic drugs which can provide clinically usefully effects with a single, once-a-day administration schedule. More particularly there is a need for dosage forms of anti-cholinergic drugs which maintain clinically effective and therapeutic serum levels of the drug over a 24 hour period to allow for once-a-day dosing, for example to treat intestinal hypermotility disorders.

### SUMMARY OF THE INVENTION

In one embodiment, the present invention is directed to a controlled release composition comprising a plurality of anti-cholinergic drug-containing particles, the particles comprising:

(a) a core comprising an anti-cholinergic drug;

(b) a first coating disposed over the core comprising at least one water-insoluble polymer; and

(c) a second coating disposed over the first coating comprising an enteric polymer optionally in combination with a water-insoluble polymer.

In another embodiment, the present invention is directed to a dosage form comprising:

(a) a core comprising an anti-cholinergic drug;

(b) a first coating disposed over the core comprising at least one water-insoluble polymer;

(c) a second coating disposed over the first coating comprising an enteric polymer optionally in combination with a water-insoluble polymer; and

(d) a plurality of rapidly-dispersing microgranules each having an average particle size of not more than about 400 μm and comprising (i) a disintegrant and (ii) a sugar alcohol and/or a saccharide, wherein said sugar alcohol and/or saccharide each have an average particle size of not more than about 30 μm;

wherein said dosage form is an orally disintegrating tablet.

In yet another embodiment, the present invention is directed to a method of preparing a controlled release composition comprising:

(a) preparing a plurality of cores comprising an anti-cholinergic drug;

(b) coating said cores with a first coating comprising at least one water-insoluble polymer optionally in combination with an enteric polymer;

(c) applying a second coating, disposed over said coated cores from step (b) wherein the second coating comprises an enteric polymer optionally in combination with a water-insoluble polymer; and

(d) filling said cores comprising an anti-cholinergic drug of step (a) and said coated cores of step (c) in clinically effective quantities into capsules.

In still yet another embodiment, the present invention is directed to a method of preparing a controlled release composition comprising:

(a) preparing a plurality of cores comprising an anti-cholinergic drug;

(b) coating said cores with a first coating comprising at least one water-insoluble polymer;

(c) applying a second coating, disposed over said coated cores from step (b) wherein the second coating comprises an enteric polymer optionally in combination with a water-insoluble polymer;

(d) preparing rapidly-dispersing microgranules each having an average particle size of not more than about 400 μm and comprising (i) a disintegrant and (ii) a sugar alcohol and/or a saccharide, wherein said sugar alcohol and/or saccharide each have an average particle size of not more than about 30 μm; and

(e) compressing a blend comprising clinically effective amounts of said cores of step (a) and said coated said drug particles of step (c), together with said rapidly-dispersing microgranules of (d) into orally disintegrating tablets.

In still another embodiment, the present invention is directed to a method of treating intestinal hypermotility or irritable bowel syndrome, comprising administering a therapeutic amount of the composition of the present invention to a patient in need thereof.

### BRIEF DESCRIPTION OF THE DRAWINGS

FIG. 1 illustrates the cross-section of embodiments of CR (controlled release) beads.

FIG. 1A: CR Bead (10) comprising a TPR (timed, pulsatile release) coating (9) disposed over a SR coated IR bead (inert core (1) coated with an anti-cholinergic drug layer (3), seal coat (5), and SR coating (7)).

FIG. 1B: CR Bead (15) comprising a DR coating (13) disposed over an SR coated IR bead (inert core (1) coated with an anti-cholinergic drug layer (3), seal coat (5), and SR coating (11)).

FIG. 1C: CR Bead (20) comprising a DR coating (19) disposed over a TPR coated IR bead (inert core (1) coated with an anti-cholinergic drug layer (3), seal coat (5), and TPR coating (17)).

FIG. 2 illustrates the dicyclomine release profiles of SR (sustained release) beads of Example 1.

FIG. 3 illustrates the dicyclomine release profiles of SR beads of Example 2.

FIG. 4 illustrates the dicyclomine release profiles of TPR (timed, pulsatile release) beads and CR (controlled release) beads of Example 3.

FIG. 5 illustrates the dicyclomine release profiles of SR beads and CR beads of Example 4.

FIG. 6 illustrates the dicyclomine release profiles of SR beads and CR beads of Example 5.

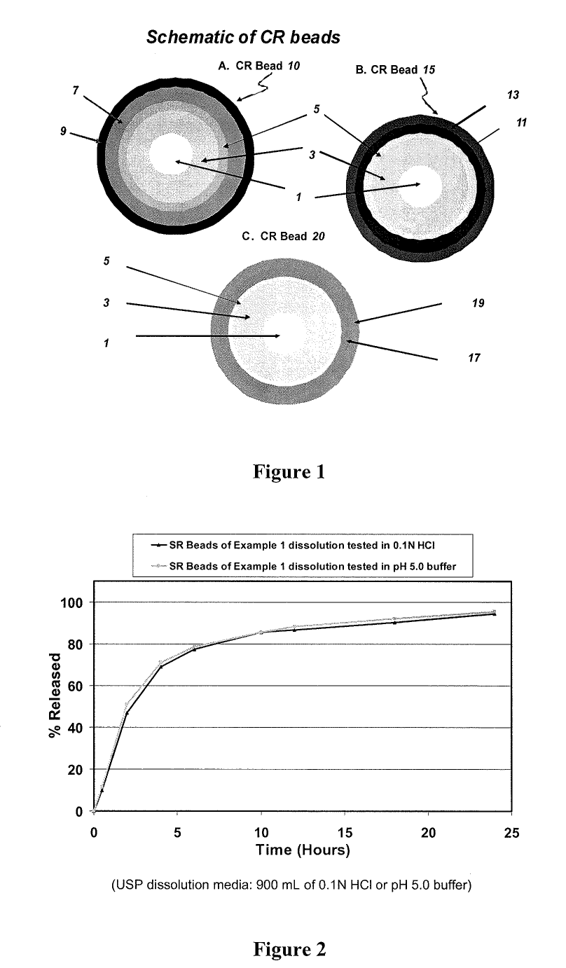

### DETAILED DESCRIPTION OF THE INVENTION

The following description includes information that may be useful in understanding the present invention. It is not an admission that any of the information provided herein is prior art or that any publication specifically or implicitly referenced is prior art.

All documents cited herein are incorporated by reference in their entirety for all purposes to the same extent as if each individual document was specifically and individually indicated to be incorporated by reference.

The terms “drug,” “active,” “active agent,” or “active pharmaceutical ingredient” as used herein include a pharmaceutically acceptable and therapeutically effective compound, pharmaceutically acceptable salts, stereoisomers and mixtures of stereoisomers, solvates (including hydrates), polymorphs, and/or esters thereof. Unless otherwise indicated, when referring to a drug in the descriptions of the various embodiments of the invention, the reference encompasses the base drug, pharmaceutically acceptable salts, stereoisomers and mixtures of stereoisomers, solvates (including hydrates), polymorphs, and/or esters thereof.

The term “salts” refers to the product formed by the reaction of a suitable inorganic or organic acid with the “free base” form of the drug. Suitable acids include those having sufficient acidity to form a stable salt, for example acids with low toxicity, such as the salts approved for use in humans or animals. Non-limiting examples of acids which may be used to form salts of dicyclomine include inorganic acids, e.g., HF, HCl, HBr, HI, H₂SO₄, H₃PO₄; non-limiting examples of organic acids include organic sulfonic acids, such as C₆₋₁₆ aryl sulfonic acids, C₆₋₁₆ heteroaryl sulfonic acids or C₁₋₁₆ alkyl sulfonic acids e.g., phenyl, α-naphthyl, β-naphthyl, (S)-camphor, methyl, ethyl, n-propyl, i-propyl, n-butyl, s-butyl, i-butyl, t-butyl, pentyl and hexyl sulfonic acids; non-limiting examples of organic acids includes carboxylic acids such as C₁₋₁₆ alkyl, C₆₋₁₆ aryl carboxylic acids and C₄₋₁₆ heteroaryl carboxylic acids, e.g., acetic, glycolic, lactic, pyruvic, malonic, glutaric, tartaric, citric, fumaric, succinic, malic, maleic, hydroxymaleic, benzoic, hydroxybenzoic, phenylacetic, cinnamic, salicylic and 2-phenoxybenzoic acids; non-limiting examples of organic acids include amino acids, e.g. the naturally-occurring amino acids, lysine, arginine, glutamic acid, glycine, serine, threonine, alanine, isoleucine, leucine, etc. Other suitable salts can be found in, e.g., S. M. Birge et al., J. Pharm. Sci., 1977, 66, pp. 1-19 (herein incorporated by reference for all purposes). In most embodiments, “salts” refers to salts which are biologically compatible or pharmaceutically acceptable or non-toxic, particularly for mammalian cells. The salts of drugs useful in the present invention may be crystalline or amorphous, or mixtures of different crystalline foul's and/or mixtures of crystalline and amorphous forms.

The terms “orally disintegrating tablet” or “ODT” refer to a tablet which disintegrates rapidly in the oral cavity of a patient after administration, without the need for chewing. The rate of disintegration can vary, but is faster than the rate of disintegration of conventional solid dosage forms (e.g., tablets or capsules) which are intended to be swallowed immediately after administration, or faster than the rate of disintegration of chewable solid dosage forms, when tested as described herein (e.g. the USP <701> test method).

The term “about” is used herein to refer to a numerical quantity, and includes “exactly.” For example, “about 60 seconds” includes 60 seconds, exactly, as well as values close to 60 seconds (e.g., 50 seconds, 55 seconds, 59 seconds, 61 seconds, 65 seconds, 70 seconds, etc.).

As used herein, the terms “controlled-release coating” and “controlled-release” encompasses coatings that delay release, sustain release, prevent release, and/or otherwise prolong the release of a drug from a particle coated with a controlled-release coating. The term “controlled-release” encompasses “sustained-release,” “timed, pulsatile release,” and “lag-time.” Thus a “controlled-release coating” encompasses a sustained release coating, timed, pulsatile release coating or “lag-time” coating.

The term “pH sensitive” as used herein refers to polymers which exhibit pH dependent solubility.

The term “enteric polymer,” as used herein, refers to a pH sensitive polymer that is resistant to gastric juice (i.e., relatively insoluble at the low pH levels found in the stomach), and which dissolves at the higher pH levels found in the intestinal tract.

As used herein, the term “immediate release” (in reference to a pharmaceutical composition which can be a dosage form or a component of a dosage form), refers to a pharmaceutical composition which in one embodiment releases greater than or equal to about 50% of the active, in another embodiment greater than about 75% of the active, in another embodiment greater than about 90% of the active, and in other embodiments greater than about 95% of the active within about 2 hours, or within about one hour following administration of the dosage form. The term can also refer to pharmaceutical compositions in which the relatively rapid release of active occurs after a “lag time” (in which little or no release of active occurs).

The term “immediate release (IR) bead” or “immediate release particle” refers broadly to an anti-cholinergic drug-containing bead or particle which exhibits “immediate release” properties with respect to the anti-cholinergic drug as described herein.

The term “sustained release (SR) bead” or “sustained release particle” refers broadly to a bead or particle comprising an SR coating, as described herein, disposed over an anti-cholinergic drug-containing core coated with an SR coating as described herein.

The term “SR coating” refers to a sustained release coating comprising a water-insoluble polymer as described herein. An SR coating by itself provides a sustained release profile.

The term “lag-time coating” or “TPR coating” refers to a controlled-release coating comprising the combination of water-insoluble and enteric polymers as used herein. A TPR coating by itself provides an immediate release pulse of the drug, or a sustained drug-release profile after a pre-determined lag time.

The term “lag-time (TPR) bead” or “lag-time particle” refers broadly to a bead or particle comprising a TPR coating, as described herein, disposed over an anti-cholinergic drug-containing core.

The term “delayed release (DR) bead” or “delayed release particle” refers broadly to an anti-cholinergic drug-containing core coated with a DR coating as described herein.

The term “DR coating” refers to a delayed release coating comprising an enteric polymer as described herein. A DR coating by itself provides a delayed release profile.

The term “controlled release (CR) bead” or “controlled release particle” refers broadly to an anti-cholinergic drug-containing core having an inner SR or TPR coating and an outer SR, DR or TPR coating as described herein.

The term “lag-time” as used herein refers to a time period wherein less than about 10% of the active is released from a pharmaceutical composition after ingestion of the pharmaceutical composition (or a dosage form comprising the pharmaceutical composition), or after exposure of the pharmaceutical composition, or dosage form comprising the pharmaceutical composition, to simulated body fluid(s), for example evaluated with a USP apparatus using a two-stage dissolution medium (first 2 hours in 700 mL of 0.1N HCl at 37° C. followed by dissolution testing at pH=6.8 obtained by the addition of 200 mL of a pH modifier).

The term “disposed over”, e.g. in reference to a coating over a substrate, refers to the relative location of e.g. the coating in reference to the substrate, but does not require that the coating be in direct contact with the substrate. For example, a first coating “disposed over” a substrate can be in direct contact with the substrate, or one or more intervening materials or coatings can be interposed between the first coating and the substrate. In other words, for example, an SR coating disposed over a drug-containing core can refer to an SR coating deposited directly over the drug-containing core, or can refer to an SR coating deposited onto a protective seal coating deposited on the drug-containing core.

The terms “plasma concentration—time profile,” “Cₘₐₓ,” “AUC,” Tₘₐₓ, and “elimination half life” have their generally accepted meanings as defined in the FDA Guidance for Industry: Bioavailability and Bioequivalence Studies for Orally Administered

Drug Products—General Considerations (issued March 2003).

Unless stated otherwise, the amount of the various coatings or layers described herein (the “coating weight”) is expressed as the percentage weight gain of the particles or beads provided by the dried coating, relative to the initial weight of the particles or beads prior to coating. Thus, a 10% coating weight refers to a dried coating which increases the weight of a particle by 10%.

In most embodiments, the present invention is directed to a controlled release composition comprising a plurality of anti-cholinergic drug-containing particles. Each of the anti-cholinergic drug-containing particles comprises a core comprising the anti-cholinergic drug. The core is coated with two or more coatings which impart the desired extended release properties. The first coating disposed over the core comprises at least one water-insoluble polymer, and the second coating disposed over the core comprises an enteric polymer and an optional water-insoluble polymer. The first and second coatings can be applied in any order. That is, the first coating can be applied over the anti-cholinergic drug-containing core particle, followed by the second coating, or the second coating can be applied to the anti-cholinergic drug-containing core particle followed by the first coating. Other coatings in addition to the first and second coating can also be applied (e.g., seal coatings or other extended release coatings) in any order, i.e., prior to, between, or after either of the first and second coatings.

Suitable anti-cholinergic drugs include, for example, atropine, benactyzine, benztropine, biperiden, butylscopolammonium bromide, cyclopentolate darifenacin, dexetimide, dicyclomine, emepronium, glycopyrrolate, hexahydrosiladifenidol, octylonium, orphenadrine, oxybutynin, oxyphenonium, pirenzepine, procyclidine, propantheline propylbenzilylcholine, quinidine, quinuclidinyl benzilate, scopolamine, tolterodine trihexyphenidyl, tropicamide, mivacurium, atracurium, doxacurium, cisatracurium, vecoronium, rocuronium, pancuronium, tabocurarine, gallamine, pipecuronium, hexamethonium, mecamylamine, trimethaphan, succinylcholine, suxamethonium, decamethonium, methoxycoronaridine, mecamylamine, imidafenacin, and the like.

In a particular embodiment, the anti-cholinergic drug of the compositions of the present invention comprises dicyclomine or salts, and/or solvates thereof. Dicylomine (bicyclohexyl-1-carboxylic acid, 2-(diethylamino) ethyl ester) refers to a compound having the following structure:

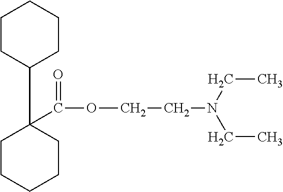

or salts, and/or solvates thereof.

In one embodiment, the anti-cholinergic drug-containing cores can take the form of anti-cholinergic drug-layered beads, pellets (e.g., extruded and spheronized compositions containing at least one anti-cholinergic drug), anti-cholinergic drug-containing granules, or anti-cholinergic drug crystals.

In another embodiment, the anti-cholinergic drug-containing core is a drug-layered bead. A drug-layered bead refers to an inert bead (e.g. a sugar sphere) coated with a drug layer, e.g., an anti-cholinergic drug layer. In other embodiments, an inert bead of the present invention can comprise microcrystalline cellulose, mannitol-microcrystalline cellulose, or silicon dioxide. The inert beads typically have particle sizes of 20-80 mesh, for example 25-30 mesh or 60-80 mesh.

An inert core thus coated with a drug layer, and lacking extended release coatings has immediate release properties, and can be referred to as an “IR bead.” Depending on the characteristics of the specific anti-cholinergic drug, the drug can be deposited from solution directly onto the inert core without using a binder. In various other embodiments, the drug layer contains a binder (typically a pharmaceutically acceptable water-soluble polymer) that facilitates the binding of the anti-cholinergic drug to the inert sugar sphere.

Examples of suitable binders include, but are not limited to, polyvinylpyrrolidone (PVP), polyethylene oxide, hydroxypropyl methylcellulose (HPMC), hydroxypropylcellulose (HPC), and polysaccharides. The binder can be present in an amount ranging from about 0.5 to about 10 weight % based on the total weight of the drug layer.

The drug layer is typically deposited by spraying a drug and optionally binder containing solution onto the inert cores, e.g., using a fluidized bed coating apparatus. The drug layering solution comprises a pharmaceutically acceptable solvent in which the anti-cholinergic drug and optional binder are dissolved. In some embodiments, the anti-cholinergic drug may be present in the form of a suspension. Depending on the viscosity, the solids content of the drug-layering solution may be up to about 35 weight %, for example about 10%, about 15%, about 20%, about 25%, about 30%, etc. Pharmaceutically acceptable solvents include water, alcohols (such as ethanol), acetone, etc.

Alternatively, the anti-cholinergic drug-containing core can be a granulate comprising the anti-cholinergic drug in combination with one or more pharmaceutically acceptable excipients (e.g., lactose, mannitol, microcrystalline cellulose, etc.). Such granulates can be prepared by conventional granulation methods, and may optionally include suitable binders as described herein.

In some embodiments, the anti-cholinergic drug-containing core of the present invention has an average particle size of not more than about 400 μm, in other embodiments not more than about 300 μm, and in yet other embodiments, not more than about 200 μm. The term “sealant layer” refers to a protective membrane disposed over a drug-containing core particle. The sealant layer protects the particle from abrasion and attrition during handling.

The term “substantially disintegrates” refers to a level of disintegration amounting to disintegration of at least about 50%, at least about 60%, at least about 70%, at least about 80%, at least about 90%, or about 100% disintegration. The term “disintegration” is distinguished from the term “dissolution”, in that “disintegration” refers to the breaking up of or loss of structural cohesion of e.g. the constituent particles comprising a tablet, whereas “dissolution” refers to the solublization of a solid in a liquid (e.g., the solublization of a drug in solvents or gastric fluids).

The compositions of the present invention comprise a plurality of anti-cholinergic drug-containing particles comprising an anti-cholinergic drug-containing core coated with a first and second coating as described herein, wherein the first coating comprises at least one water-insoluble polymer. The first coating can be disposed directly on the anti-cholinergic drug-containing core, coated onto a sealant layer which is disposed over the drug-containing core, coated over the second coating, coated over a sealant layer which is disposed over the second coating, etc.

The term “water-insoluble polymer” refers to a polymer which is insoluble or very sparingly soluble in aqueous media, independent of pH, or over a broad pH range (e.g., pH 1.0 to pH 14). A polymer that swells but does not dissolve in aqueous media can be “water-insoluble,” as used herein.

The term “water-soluble polymer” refers to a polymer which is soluble (i.e., a significant amount dissolves) in aqueous media, independent of pH.

The term “enteric polymer” refers to a polymer which is soluble (i.e., a significant amount dissolves) under intestinal conditions (i.e., in aqueous media under neutral to alkaline conditions) and is insoluble under acidic conditions (i.e., low pH).

The term “reverse enteric polymer” refers to a polymer that is soluble under acidic conditions and insoluble under neutral and alkaline conditions.

In one embodiment, the first coating comprising the water-insoluble polymer (but no optional enteric polymer) is coated onto the anti-cholinergic drug-containing core (wherein the core is optionally coated with a sealant layer), thereby providing a sustained release (SR) coating.

Non-limiting examples of suitable water-insoluble polymers include ethylcellulose, cellulose acetate, cellulose acetate butyrate, polyvinyl acetate, neutral copolymers of acrylate/methacrylate esters (e.g., Eudragit NE, which is a copolymer of ethyl acrylate and methyl methacrylate), waxes, and mixtures thereof. In a particular embodiment, the water-insoluble polymer comprises ethylcellulose. In another particular embodiment, the water-insoluble polymer comprises ethylcellulose with a mean viscosity of 10 cps in a 5% solution in 80/20 toluene/alcohol measured at 25° C. on an Ubbelohde viscometer. Suitable coating weights for a first coating comprising a water-insoluble polymer range from about 3% to about 40%, including about 3%, about 5%, about 7%, about 10%, about 12%, about 15%, about 17%, about 20%, about 22%, about 25%, about 27%, about 30%, about 35%, and about 40%, inclusive of all ranges and subranges therebetween.

In some embodiments, the water-insoluble polymer of the SR coating provides suitable properties (e.g., extended release characteristics, mechanical properties, and coating properties) without the need for a plasticizer. For example, coatings comprising polyvinyl acetate (PVA), neutral copolymers of acrylate/methacrylate esters, ethylcellulose, waxes, etc. can be applied without plasticizers.

In yet another embodiment, the water-insoluble polymer of the SR coating may include a plasticizer. The amount of plasticizer required depends upon the plasticizer, the properties of the water-insoluble polymer, and the ultimate desired properties of the coating. Suitable levels of plasticizer range from about 1% to about 20%, from about 3% to about 20%, about 3% to about 5%, about 7% to about 10%, about 12% to about 15%, about 17% to about 20%, or about 1%, about 2%, about 3%, about 4%, about 5%, about 6%, about 7%, about 8%, about 9%, about 10%, about 15%, or about 20% by weight relative to the total weight of the coating, inclusive of all ranges and subranges therebetween.

Non-limiting examples of suitable plasticizers include triacetin, citrate esters, triethyl citrate, acetyltriethyl citrate, tributyl citrate, acetyl tri-n-butyl citrate, diethyl phthalate, dibutyl phthalate, dioctyl phthalate, methyl paraben, propyl paraben, propyl paraben, butyl paraben, dibutyl sebacate, substituted triglycerides and glycerides, monoacetylated and diacetylated glycerides (e.g., Myvacet® 9-45), glyceryl monostearate, glycerol tributyrate, polysorbate 80, polyethylene glycol, propylene glycol, oils (e.g. castor oil, hydrogenated castor oil, rape seed oil, sesame oil, olive oil, etc.), glycerin sorbitol, diethyl oxalate, diethyl malate, diethyl fumarate, diethylmalonate, dibutyl succinate, fatty acids, and mixtures thereof.

Further non-limiting examples of suitable plasticizers include glycerol and esters thereof (e.g., monoacetylated glycerides, acetylated mono- or diglycerides (e.g., Myvacet® 9-45)), glyceryl monostearate, glyceryl triacetate, glyceryl tributyrate, phthalates (e.g., dibutyl phthalate, diethyl phthalate, dimethyl phthalate, dioctyl phthalate), citrates (e.g., acetylcitric acid tributyl ester, acetylcitric acid triethyl ester, tributyl citrate, acetyltributyl citrate, triethyl citrate), glyceroltributyrate; sebacates (e.g., diethyl sebacate, dibutyl sebacate), adipates, azelates, benzoates, chlorobutanol, polyethylene glycols, vegetable oils, fumarates, (e.g., diethyl fumarate), malates, (e.g., diethyl malate), oxalates (e.g., diethyl oxalate), succinates (e.g., dibutyl succinate), butyrates, cetyl alcohol esters, malonates (e.g., diethyl malonate), castor oil, and mixtures thereof. When used in an embodiment of the present invention, the plasticizer may constitute from about 3% to about 30% by weight of the polymer(s) in the controlled-release coating. In still other embodiments, the amount of plasticizer relative to the weight of the polymer(s) in the controlled-release coating is about 3%, about 5%, about 7%, about 10%, about 12%, about 15%, about 17%, about 20%, about 22%, about 25%, about 27%, and about 30%, inclusive of all ranges and subranges therebetween. One of ordinary skill in the art will recognize that the presence of plasticizer, or type(s) and amount(s) of plasticizer(s) can be selected based on the polymer or polymers and nature of the coating system (e.g., aqueous or solvent-based, solution or dispersion-based and the total solids).

In yet another embodiment, the first coating can comprise a combination of the water-insoluble polymer with a water-soluble polymer. In one embodiment, the ratio of the water-insoluble polymer to the water-soluble polymer ranges from about 95:5 to about 50:50, including the range from about 90:10 to about 60:40, or about 90:10, about 85:15, about 80:20, about 75:25, about 70:30, about 65:35, about 60:40, about 55:45, or about 50:50, inclusive of all values, ranges and subranges therebetween.

In one embodiment, the coating weight of a first coating comprising a combination of water-insoluble and water-soluble polymers ranges from about 3% to about 50% by weight, including about 10% to about 40%, about 20% to about 30%, or about 3%, about 4%, about 5%, about 6%, about 7%, about 8%, about 9%, about 10%, about 12%, about 14%, about 60%, about 18%, about 20%, about 25%, about 30%, about 35%, about 40%, about 45%, or about 50%, inclusive of all ranges and subranges therebetween. In other embodiments, the coating weight of a first coating comprising water-insoluble and water-soluble polymers in combination is about 3% to about 5%, about 7% to about 10%, about 12% to about 15%, about 17% to about 20%, about 22% to about 25%, about 27% to about 30%, about 35% to about 40%, or about 45% to about 50% of the weight of the coated core, inclusive of all values, ranges and subranges therebetween.

Suitable water-soluble polymers include but are not limited to polyvinylpyrrolidone (e.g., Povidone K-25), polyethylene glycol (e.g., PEG 400), hydroxypropyl methylcellulose, and hydroxypropylcellulose.

In various embodiments, the second coating layer comprises an enteric polymer in combination with an optional water-insoluble polymer. When the second coating comprises both an enteric polymer and a water-insoluble polymer, a timed pulsatile release (TPR) coating is provided. In still other embodiments, when the second coating comprises an enteric polymer (without the water-insoluble polyer) disposed on the anti-cholinergic drug-containing particle, a delayed release (DR) coating is provided.

Non-limiting examples of suitable enteric polymers include cellulose acetate phthalate, hydroxypropyl methylcellulose phthalate, hydroxypropyl methylcellulose acetate succinate, polyvinyl acetate phthalate, pH-sensitive methacrylic acid/methylmethacrylate copolymers (e.g., Eudragit® L, S and FS polymers), shellac, and mixtures thereof. In certain embodiments, non-polymeric enteric materials such as non-polymeric waxes and fatty acid compositions may be used instead of enteric polymers, provided they have the pH sensitive solubility associate with enteric polymers. These enteric polymers may be used as a solution in a solvent mixture or an aqueous dispersion. Some commercially available materials that may be used are methacrylic acid copolymers sold under the trademark Eudragit (L100, 5100, L30D) manufactured by Rohm Pharma, Cellacefate (cellulose acetate phthalate) from Eastman Chemical Co., Aquateric (cellulose acetate phthalate aqueous dispersion) from FMC Corp., and Aqoat (hydroxypropyl methylcellulose acetate succinate aqueous dispersion) from Shin Etsu K.K.

When the second coating comprises a water-insoluble polymer in combination with the enteric polymer (e.g., a TPR coating), the ratio of the water-insoluble polymer to enteric polymer ranges from about 10:1 to about 1:1, including the ranges of from about 9:1 to about 3:1, and from about 3:1 to about 1:1. In particular embodiments, the ratio of water-insoluble polymer to enteric polymer is about 1:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, or about 10:1, inclusive of all values, ranges, and subranges therebetween. In most embodiments of the compositions of the present invention having a TPR coating, the TPR coating is applied at a coating weight of about 5% to about 60% by weight, including the ranges of from about 10% to about 50%, from about 20% to about 40%, and from about 25% to about 35%, or at a coating weight of about 5%, about 6%, about 7%, about 8%, about 9%, about 10%, about 12%, about 14%, about 16%, about 18%, about 20%, about 25%, about 30%, about 35%, about 40%, about 45%, or about 50%, inclusive of all ranges and subranges therebetween.

In a particular embodiment, the TPR coating comprises ethylcellulose (e.g., EC-10) as the water-insoluble polymer and hypromellose phthalate (e.g., HP-55) as the enteric polymer.

Similar to the SR coating, DR and TPR coatings can be plasticizer-free or also include one or more optional plasticizers (e.g. any of the plasticizers described herein). The amount of plasticizer required, when present, depends upon the plasticizer, the properties of the water-insoluble and/or enteric polymer(s), and the ultimate desired properties of the coating. Suitable levels of plasticizer range from about 1% to about 20%, from about 3% to about 20%, about 3% to about 5%, about 7% to about 10%, about 12% to about 15%, about 17% to about 20%, or about 1%, about 2%, about 3%, about 4%, about 5%, about 6%, about 7%, about 8%, about 9%, about 10%, about 15%, or about 20% by weight relative to the total weigh of the coating, inclusive of all ranges and subranges therebetween.

As described herein, in various embodiments the controlled release compositions of the present invention comprise a plurality of anti-cholinergic drug-containing particles, coated with a first coating of an SR layer (comprising a water-insoluble polymer, or the combination of a water-insoluble polymer and a water-soluble polymer), then a second coating of a DR or a TPR layer (comprising an enteric polymer or the combination of an enteric and a water-insoluble polymer, respectively). In various alternative embodiments, the controlled release compositions of the present invention comprise a plurality of anti-cholinergic drug-containing particles, coated with a first coating of an SR layer (as described herein) or a TPR layer (comprising a water-insoluble polymer and an enteric polymer), then a second coating of a DR layer (comprising an enteric polyer without a water-insoluble polymer). In a particular embodiment, the controlled release compositions of the present invention comprise a plurality of anti-cholinergic drug-containing particles, coated with a first coating of a TPR layer (comprising a water-insoluble polymer and an enteric polymer) and a second coating of a DR layer (comprising an enteric polymer without a water-insoluble polymer).

In other particular embodiment, the controlled release compositions of the present invention comprise a plurality of drug-containing core particles. The drug can be an anti-cholinergic drug as described herein, but in other particular embodiments, the drug is not restricted to anti-cholinergic drugs as described herein, but can include other suitable classes of drugs known in the pharmaceutical arts. In this particular embodiment the core particles comprise any of the types of core particles described herein (e.g., granules, drug layered beads, drug crystals, etc., optionally seal coated with a sealant layer as described herein) coated with an inner SR layer (e.g., ethylcellulose, optionally plasticized), and an outer DR layer (e.g. the hydroxypropylmethylcellulose phthalate, optionally plasticized).

In another particular embodiment, the controlled release compositions of the present invention may comprise an active pharmaceutical ingredient selected from the following non-limiting examples of drug classes: analgesics (e.g., ibuprofen, sulindac, celecoxib, meloxicam), anticonvulsants (e.g., lorazepam, pregabalin, ritagabine), antidiabetic agents (e.g., glipizide, ripaglinide, pioglitazone), anti-infective agents (e.g., mefloquine, cifrofloxacin, cefuroxime, ceftriaxone, metronidazole), anti-Parkinsonian agents (e.g., selegiline, pramipexole, ropinirole), antirheumatic agents (e.g., azathioprine), cardiovascular agents (e.g., carvedilol, sotalol, pindolol), central nervous system (CNS) stimulants (e.g., alprazolam, methylphenidate, amphetamines), dopamine receptor agonists (e.g., aripiprazole, olanzapine, ziprasidone), anti-emetics (e.g., ondansetron, mirtazapine, dolasetron, domperidone), gastrointestinal agents (e.g., cisapride, pantoprazole, ranitidine), psychotherapeutic agents (e.g., antipsychotics such as clozapine, iloperidone, perphenazine), opioid agonists (e.g., papaverine, oxymorphone, hydromorphone), opioid antagonists (e.g., oxycodone, buprenorphine), anti-epileptic drugs (lamotrigine, midazolam, tiagabine), histamine H₂ antagonists (e.g., famotidine), anti-asthmatic agents (e.g., metaproterenol, salbutamol, theophylline), and skeletal muscle relaxants (e.g., cyclobenzaprine, metaxalone, clonidine).

In still another particular embodiment, the controlled release compositions of the present invention comprise a plurality of anti-cholinergic drug-containing particles (e.g., drug-layered inert cores, optionally seal coated with a sealant layer as described herein), coated with an inner SR layer (e.g., ethylcellulose, optionally plasticized), and an outer TPR layer (e.g., ethylcellulose and hydroxypropylmethylcellulose phthalate, optionally plasticized).

In yet still another particular embodiment, the controlled release compositions of the present invention comprise a plurality of anti-cholinergic drug-containing particles (e.g. drug-layered inert cores, optionally seal coated with a sealant layer as described herein), coated with an inner TPR layer (e.g., ethylcellulose and hydroxypropylmethylcellulose phthalate, optionally plasticized), and an outer DR layer (e.g. the hydroxypropylmethylcellulose phthalate, optionally plasticized).

In particular embodiments, the DR layer comprises plasticized hydroxypropyl methylcellulose phthalate (e.g. HP-55 and triethyl citrate). In more particular embodiments, the DR layer comprises about 90/10 HP-55/triethyl citrate.

In particular embodiments the SR layer comprises plasticized ethylcellulose (e.g. EC-10 and triethyl citrate). In more particular embodiments, the SR layer comprises about 90/10 EC-10 and triethyl citrate.

In particular embodiments the TPR layer comprises a plasticized mixture of hydroxypropylmethylcellulose phthalate and ethylcellulose (e.g. HP-55/EC-10 and triethyl citrate). In more particular embodiments, the TPR layer comprises HP-55/EC-10 containing approximately 10% triethyl citrate.

The extended release compositions of the present invention may further comprise a sealant layer disposed on the anti-cholinergic drug-containing particle, e.g. between the first and second coatings, beneath the first and second coatings, and/or over both of the first and second coatings to prevent (or minimize) static and/or particle attrition during processing and handling.

In one embodiment, the sealant layer comprises a hydrophilic polymer. Non-limiting examples of suitable hydrophilic polymers include hydrophilic hydroxypropylcellulose (e.g., Klucel® LF), hydroxypropyl methylcellulose or hypromellose (e.g., Opadry® Clear or Pharmacoat™ 603), vinylpyrrolidone-vinylacetate copolymer (e.g., Kollidon® VA 64 from BASF), and ethylcellulose, e.g. low-viscosity ethylcellulose. The sealant layer can be applied at a coating weight of about 1% to about 10%, for example about 1%, about 2%, about 3%, about 4%, about 5%, about 6%, about 7%, about 8%, about 9%, or about 10%, inclusive of all ranges and subranges therebetween.

In one embodiment, a controlled release composition of the present invention comprises an anti-cholinergic drug-layered inert sugar bead coated with individual or multiple controlled release coatings.

In another embodiment, the compositions of the present invention further comprise a compressible coating disposed over the controlled-release coating (or disposed on the outer-most coating, if the controlled-release coating is further coated with a coating with an enteric polymer). The compressible coating comprises a polymer, including but not limited to hydroxypropylcellulose, poly(vinyl acetate-vinyl pyrrolidone), polyvinyl acetate, ethylcellulose (e.g., plasticized low-viscosity ethylcellulose latex dispersions), etc. The compressible coating can be plasticized or unplasticized, and promotes the integrity of the controlled-release coating during compression.

In another embodiment controlled release compositions of the present invention can further comprise rapidly disintegrating granules comprising a saccharide and/or a sugar alcohol in combination with a disintegrant. Suitable disintegrants include, but are not limited to for example, disintegrants selected from the group consisting of crospovidone, sodium starch glycolate, starch, crosslinked sodium carboxymethylcellulose, low-substituted hydroxypropylcellulose, gums (e.g., gellan gum) and combinations thereof. Suitable saccharides and/or sugar alcohols may be selected from the group consisting of arabitol, erythritol, glycerol, hydrogenated starch hydrolysate, isomalt, lactitol, lactose, maltitol, mannitol, sorbitol, xylitol, sucrose, maltose, and combinations thereof. The saccharide and/or sugar alcohol may also be supplemented or replaced with artificial sweeteners such as sucralose. The ratio of the disintegrant to the saccharide and/or sugar alcohol in the rapidly dispersing microgranules ranges from about 1:99 to about 10:90, from about 5:95 to about 10:90 on a weight basis and inclusive of all ranges and subranges therebetween. In most embodiments, the disintegrant or the saccharide and/or sugar alcohol, or both, are present in the form of particles having an average particle size of about 30 μm or less. The ratio of the anti-cholinergic drug-containing beads to the rapidly disintegrating granules can range from about 1:6 to about 1:2, from about 1:5 to about 1:3, or about 1:6, about 1:5, about 1:4, about 1:3, or about 1:2, inclusive of all ranges and subranges therebetween.

The multiple controlled-release coatings of the compositions of the present invention contribute to the control of dissolution at the drug interface and hence control the release of the anti-cholinergic drug (e.g. dicyclomine or salts, and/or solvates thereof) from the particles of the controlled release compositions of the present invention. The achievable lag time, delayed release time, or sustained-release properties depend on the composition and thickness of the controlled-release coatings. Specific factors that can affect achieving optimal once-daily dosage forms include, but are not limited to, the pKa of the anti-cholinergic drug and its solubility, e.g. in GI fluids.

The in vitro drug release data obtained particles coated with the multiple controlled release coatings described herein provide release profiles for an anti-cholinergic drugs, which thereby provide pharmacokinetic profiles suitable for a once- or twice-daily dosing regimens. In one embodiment, the sustained-release coating provides release of an anti-cholinergic drug which is sustained over about 8-12 (twice daily) to about 16-20 hours (once daily) when tested in the two-stage dissolution method (700 mL of 0.1N HCl (hydrochloric acid) for the first 2 hours and thereafter in 900 mL at pH 6.8 obtained by adding 200 mL of a pH modifier), suitable for a once- or twice-daily dosing regimen. For example, a suitable release profile for the controlled release particles of the present invention substantially corresponds to the following pattern when dissolution tested using United States Pharmacopoeia Apparatus 2 (paddles @ 50 rpm) in a 2-stage dissolution media (700 mL of 0.1N HCl for the first 2 hrs followed by testing in 900 mL buffer at pH 6.8 obtained by adding 200 mL of a pH modifier) at 37° C.:
after 4 hours, about 40±20% of the total anti-cholinergic drug is released;
after 8 hours, about 65±25% of the total anti-cholinergic drug is released; and
after 12 hours, about 70±30% of the total anti-cholinergic drug is released.

The controlled release compositions of the present invention can be formulated with optional pharmaceutically acceptable excipients (binders, a disintegrants, fillers, diluents, compression aids (e.g., microcrystalline cellulose/fused silicon dioxide), lubricants, etc.) into any suitable oral dosage form, for example sachets, tablets, capsules, or orally disintegrating tablets (ODTs). In one embodiment, the dosage form is a tablet, for example a tablet with a friability of less than about 1%. In another embodiment, the dosage form is a capsule filled with at least one population of particles comprising the controlled release composition of the present invention. The capsule can be for example, a gelatin capsule, or an HPMCP (hydroxypropylmethylcellulose) capsule.

In other embodiments, the dosage form is an ODT. ODTs of the present invention disintegrate in the oral cavity, and are easily swallowed without water. For example, an ODT of the present invention substantially disintegrates within about 60 seconds after contact with saliva in the oral cavity or with simulated saliva fluid. In another embodiment, the ODT substantially disintegrates within about 30 seconds. Disintegration is tested according to the USP <701> Disintegration Test (herein incorporated by reference in its entirety for all purposes). In most embodiments, the ODT substantially disintegrates in the oral cavity of a patient, forming a smooth, easy-to-swallow suspension having no gritty mouthfeel or aftertaste, and provides a target PK profile (e.g., plasma concentration vs. time plot) of the anti-cholinergic drug (e.g., dicyclomine) suitable for a once- or twice-daily dosing regimen.

For example, the ODT provides prolonged release of the anti-cholinergic drug over a period of 8-20 hrs, which substantially corresponds to the pattern disclosed above, although somewhat broader release ranges at 4, 8, and 12 hours may be suitable in certain embodiments.

ODT formulations of the present invention are especially useful for treating geriatric patients (who often have difficulty swallowing conventional tablets and capsules) or for treating mentally ill patients (who often resist or “cheek” their medications). The administration of ODTs to geriatric and/or mentally ill patients will reduce the frequency of dosing and ease patient non-compliance issues.

In a particular embodiment, the ODT of the present invention comprises a therapeutically effective amount of dicyclomine or salts and/or solvates thereof. After administration, the ODT substantially disintegrates in the oral cavity of a patient, forming a smooth, easy-to-swallow suspension having no gritty mouthfeel or aftertaste, and provides a target PK profile (i.e., plasma concentration vs. time plot) of dicyclomine suitable for a once- or twice-daily dosing regimen. In addition to the controlled release composition of the present invention and rapidly disintegrating granules, the ODT of the present invention optionally includes pharmaceutically acceptable excipients such as compressible diluents, fillers, coloring agents, and optionally a lubricant.

The dosage forms of the present invention can comprise two or more populations of anti-cholinergic drug-containing particles, including at least one population of controlled release particles as described herein. For example, the dosage form can comprise a population of controlled release particles as described herein, and in addition, immediate release (IR) particles, for example uncoated cores comprising an anti-cholinergic drug. In one embodiment, the dosage form comprising two or more populations of anti-cholinergic drug-containing particles is an ODT. When the dosage form is ODT, the two or more populations of anti-cholinergic drug-containing particles are combined with rapidly disintegrating microgranules, and the anti-cholinergic drug-containing particles and rapidly disintegrating microgranules have a particle size which provides a smooth, non-gritty mouth feel after disintegration of the ODT in the oral cavity. In one embodiment, an ODT of the present invention comprises one of SR, DR or CR particle populations; in another embodiment, the ODT comprises a combination of IR particles and SR particles; in yet another embodiment, the ODT comprises SR particles in combination with enteric coated TPR particles, and optionally in combination with (optionally taste-masked) IR particles (in addition to rapidly disintegrating microgranules). In yet another embodiment, an ODT of the present invention comprises: enteric coated SR beads with or without a compressible coating in combination with rapidly dispersing granules (e.g., mannitol-crospovidone microgranules).

If the ODT of the present invention includes IR particles, the IR particles can be coated with a taste-masking coating which allows immediate release of the anti-cholinergic drug but prevents release in the oral cavity, and thus any off-taste from the anti-cholinergic drug. That is, a taste-masked IR particle releases not more than about 10% of the total amount of anti-cholinergic drug contained in the IR particle in 3 minutes (the longest typical residence time anticipated for the ODT in the buccal cavity) when dissolution tested in simulated saliva fluid (pH˜6.8), while releasing not less than about 75% of the total amount anti-cholinergic drug in the IR particles in about 60 minutes when dissolution tested in 0.1N HCl.

In various embodiments of the present invention, when the dosage form comprises IR particles in addition to the controlled release particles, the ratio of IR particles to SR and/or TPR particles ranges from about 0:100 (i.e., no IR particles) to about 50:50, for example from about 10:90 to about 20:80, from about 30:70 to about 40:60, or about 5:95, about 10:90, about 15:85, about 20:80, about 25:75, about 30:70, about 35:65, about 40:60, about 45:55, or about 50:50, inclusive of all ranges and subranges therebetween.

In a particular embodiment of the dosage forms of the present invention, the dosage forms comprise dicyclomine or salts, polymorphs, and/or solvates thereof (including hydrates).

In other embodiments of the present invention, the plurality of beads in a dosage form can yield different desired anti-cholinergic drug (e.g., dicyclomine) release profiles. In one embodiment, for example, a once-daily dosage form comprising dicyclomine with an elimination half-life of about 2 hours may contain a mixture of a population of taste-masked IR particles (which provides an immediate-release pulse of the anti-cholinergic drug), an SR particle population with an enteric or TPR coating), which exhibits the target release profile over about 8-20 hours, and maintains clinically effective plasma concentrations of the anti-cholinergic drug at 12-24 hours.

In another embodiment, the present invention is directed to methods of preparing a controlled release composition comprising the step of (a) preparing a core comprising an anti-cholinergic drug; (b) applying a first coating comprising at least one water-insoluble polymer over the core; (c) applying a second coating comprising an enteric polymer optionally in combination with a water-insoluble polymer; wherein the first and second coatings can be applied in any order.

The step of preparing the core may be accomplished by any of the methods known in the art; for example, layering an inert bead (e.g., sugar, microcrystalline cellulose, mannitol-microcrystalline cellulose, silicon dioxide, etc.) with a solution comprising the drug and optionally a polymeric binder (e.g., by fluid-bed or pan coating), or by granulating particles of the drug with optional excipients, or by extrusion and spheronization, etc. Alternatively, “preparing a core” can comprise obtaining or preparing drug particles or crystals of the desired particle size (e.g., about 50-500 μm, including 100-250 μm).

In some embodiments, the method comprises preparing core particles comprising the anti-cholinergic drug (as described herein), then coating the core particles with an SR coating (as described herein), followed by a TPR coating (as described herein) or a DR coating (as described herein). In other embodiments, the method comprises preparing core particles comprising the anti-cholinergic drug, and then coating the core particles with a TPR or DR coating, followed by an SR coating. In still other embodiments, the method comprises preparing core particles comprising the anti-choliergic drug, and then coating the core particles with an SR or TPR coating, followed by a DR coating. For each of these embodiments, optional sealant layers can be applied under, over, and/or between the controlled-release layers.

In yet another embodiment, the method of the present invention further comprises blending the controlled-release composition described herein with the rapidly dispersing granules described herein, and compressing the blended controlled-release composition and rapidly dispersing granules into an ODT.

In another embodiment, the method further comprises coating a compressible layer comprising a hydrophilic polymer (e.g., hydroxypropylcellulose), over the controlled-release layers to eliminate/minimize damage to the extended-release coating(s) of the extended-release particles during compression into an ODT.

In yet another embodiment, the method of the present invention further comprises blending the controlled-release composition described herein with optional excipients, and compressing the blended composition and optional excipients into a tablet.

In still yet another embodiment, the method of the present invention further comprises filling a capsule with the controlled-release composition described herein and optional excipients. Suitable capsules include, for example, hard gelatin capsules and HPMCP capsules.

In a particular embodiment, the method of the present invention comprises the steps of:

(a) preparing anti-cholinergic drug particles (crystals, microgranules, drug layered beads, or pellets with an average particle size of 50-400 μm, or about 100-300 μm) comprising dicyclomine or salts, polymorphs and/or solvates thereof and optionally applying a protective seal-coat onto the drug-layered particles, thereby forming IR beads;

(b) applying a sustained-release (SR) coating comprising a water-insoluble polymer onto the IR beads at a coating weight of from about 15% to 30%, thereby framing SR beads;

(c1) applying a delayed-release (DR) coating comprising an enteric polymer onto the SR beads at a coating weight of from about 10% to 30%, thereby forming controlled-release (CR) beads; and/or

(c2) applying a lag-time (TPR) coating comprising the combination of a water-insoluble polymer and an enteric polymer at a weight ratio of from about 10:1 to 1:4 onto SR beads, at a coating weight of from about 10% to 60%, thereby forming controlled-release beads;

(d) preparing rapidly dispersing granules comprising a sugar alcohol, a saccharide, or a mixture thereof and a disintegrant;

(e) blending the controlled-release beads of step (c1) and/or step (c2) with the rapidly dispersing granules of step (d);

(f) compressing the blend of step (e), thereby forming an ODT.

In some embodiments, blending step (e) includes blending the controlled-release beads of step (c1) and/or step (c2) with optional pharmaceutically acceptable excipients (e.g., a flavor, a sweetener, a disintegrant, microcrystalline cellulose, etc.)

In other embodiments, blending step (e) includes blending the controlled-release beads of step (c1) and/or step (c2) with IR beads optionally taste-masked with a taste-masking coating comprising a water-insoluble polymer, or comprising a water-insoluble polymer in combination with a gastrosoluble organic, inorganic, or polymeric pore-former, wherein the taste masking layer substantially masks the taste of the IR beads.

In another embodiment, the ODT prepared according to the method described above substantially disintegrates within about 60 seconds after contact with saliva in the oral cavity or simulated saliva fluid. In another embodiment, the ODT prepared according to the method described above substantially disintegrates within about 30 seconds after contact with saliva in the oral cavity or simulated saliva fluid.

The term “substantially masks the taste” in reference to the taste-masking layer of the IR particles (when present) refers to the ability of the taste-masking layer to substantially prevent release of a bitter tasting drug in the oral cavity of a patient. A taste-masking layer which “substantially masks” the taste of the drug typically releases less than about 10% of the drug in the oral cavity of the patient, in other embodiments, less than about 5%, less than about 1%, less than about 0.5%, less than about 0.1%, less than about 0.05%, less than about 0.03%, less than about 0.01% of the drug. The taste-masking properties of the taste-masking layer of the compositions of the present invention can be measured in vivo (e.g., using conventional organoleptic testing methods known in the art) or in vitro (e.g., using dissolution tests as described herein). The skilled artisan will recognize that the amount of drug release associated with a taste-masking layer than “substantially masks” the taste of a drug is not limited to the ranges expressly disclosed herein, and can vary depending on other factors, such as the perceived the bitterness of the drug and the presence of other flavoring agents in the composition.

The present invention is described in greater detail in the sections below. The following examples are used to illustrate the present invention.

It should be understood that the examples and embodiments described herein are for illustrative purposes only and that various modifications or changes in light thereof will be suggested to persons skilled in the art and are to be included within the spirit and purview of this application.

### EXAMPLES

### Example 1A

IR Beads (drug load: approximately 20% as dicyclomine hydrochloride): Dicyclomine hydrochloride (700 g) was slowly added to ethanol (2100 g) until dissolved under constant stirring for not less than 10 min, and then water (700 g) was added so that the ratio of ethanol to water in the resulting solution was 75/25. A Glatt GPCG 3 equipped with a 7″ bottom spray Wurster 8″ high column, partition column gap of 15 mm from the ‘B’ bottom air distribution plate covered with a 200 mesh product retention screen (0.8 mm port nozzle) was charged with 2800 g of 60-80 mesh sugar spheres and sprayed with the dicyclomine solution (20% solids) at an initial rate of 5 g/min with a stepwise increase to 15.5 g/min, at an inlet air volume of 90-105 m³/hr, air atomization pressure of 1.50 bar while maintaining the product temperature of 37±3° C. Following rinsing of the spray system with 50 g of ethanol, the drug-layered beads were dried in the Glatt unit for 50 min to drive off residual solvents (including moisture). The resulting dicyclomine IR beads were sieved through 35 and 120 mesh screens to discard oversized particles and fines.

### Example 1B

IR Beads (drug load: approximately 20% as dicyclomine hydrochloride, with binder): Povidone (PVP K30; 100.0 g) was slowly added to 75/25 95% ethanol /water (2325.0 g of 95% ethanol and 775.0 g of water) until dissolved under constant stirring for not less than 10 min. Dicyclomine hydrochloride (800.0 g) was slowly added while stirring, until dissolved. The IR beads were prepared as described above. The IR bead batch comprising the binder (2.5% by weight) had a higher potency (>19.2% by weight) compared to the IR bead batch prepared without the polymeric binder (Example 1A). Three more bead batches were also prepared with the binder at 4.0% by weight. The four bead batches containing the binder were blended together to provide IR beads with a mean potency of 19.7% by weight dicyclomine hydrochloride.

### Example 1C

SR Beads (drug load: approximately 20% as dicyclomine hydrochloride): Ethylcellulose (EC-10, Ethocel Premium 10 from Dow Chemicals; 159.1 g) was slowly added to 95% ethanol while stirring constantly until dissolved. Triethyl citrate (TEC; 15.9 g) was slowly added until dissolved. A Glatt GPCG 1 equipped with a 6″ bottom spray Wurster 6″ high column, ‘B” bottom air distribution plate covered with a 200 mesh product retention screen, 0.8 mm port nozzle, was charged with 700 g of IR beads from Example 1A, above. The IR beads were sprayed with the SR functional polymer coating formulation (10% solids) at a product temperature of 33±3° C., atomization air pressure of 1.50 bar, inlet air flow of 50-75 m³/hr, and an initial flow rate of 1 g/min with a stepwise increase to 6 g/min for a SR coating weight of 20%. Following spraying, the coated beads were dried in the Glatt unit for 30 min to drive off residual solvents (including moisture). The resulting SR beads were sieved to provide particles having a mean particle size of less than about 500 μm.

FIG. 2 shows the drug release profiles for SR beads tested in acidic and pH 5.0 buffers. No difference was observed in the release profile of SR beads at dissolution times of up to 24 hours. About 70% of the drug was released in 4 hrs.

### Example 2

SR Beads (coating level: approximately 30%): Ethylcellulose (214.3 g) was slowly added to a 90/10 mixture of acetone (1716.5 g) and water (190.8 g) while stirring constantly until dissolved. Triethyl citrate (21.4 g) was slowly added to dissolve. The IR beads (500 g) from 1A, above, were fluid-bed coated (Glatt 1 with 4″ Wurster insert, 15 cm high) with the functional polymer solution (11% solids) at a product temperature of 33±3° C., atomization air pressure of 1.50 bar, inlet air flow of 50-75 m³/hr, and a spray flow rate of 3.0-6.0 g/min for a SR coating weight of 30%. The resulting SR beads were dried in the Glatt unit for 30 min to drive off residual solvents. About 85% by weight of the coated beads had mean particle size of less than about 355 μm (Example 2A). Another batch of SR beads (Example 2B) was similarly prepared using the IR beads (500 g from Example 1A, above) by spraying a less concentrated polymer solution (i.e., at 5.5% solids). About 99% of the coated pellets collected by sieving had a mean particle size of less than about 355 μm. In both cases, samples were pulled at 20% coating weights for analytical testing (e.g., assay and drug release).

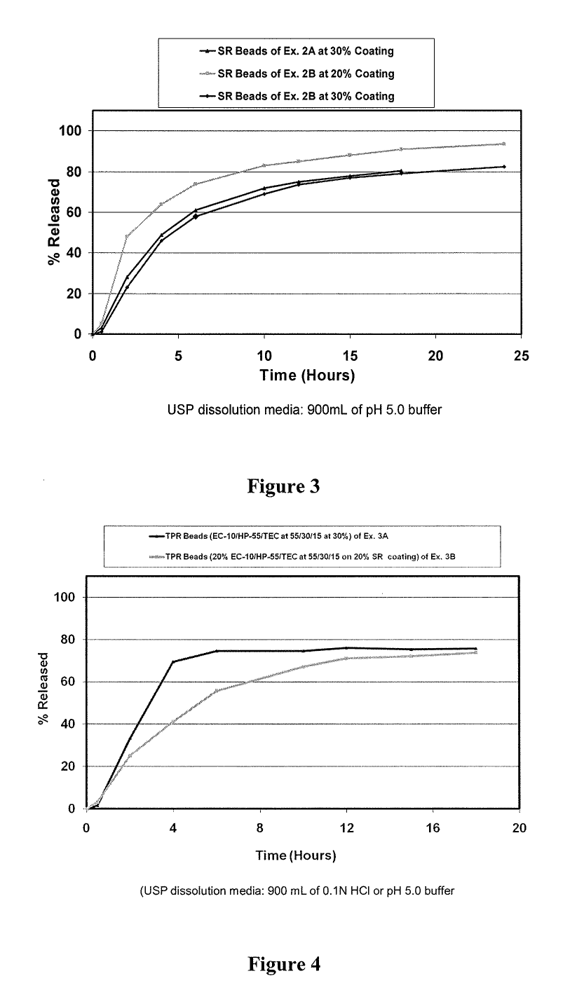

FIG. 3 shows the drug release profiles for SR beads from Example 2A at a coating weight of 30%, and SR beads from Example 2B at 20% and 30% coating weights.

### Example 3A

TPR Beads (EC-10/HP-55/TEC at 55/30/15): Ethylcellulose (EC-10; 93.0 g) was slowly added to acetone/water at 90/10 (1876.4 g of acetone and 208.5 g of water) while stirring rigorously to dissolve. Hypromellose phthalate (HP-55 from Shin Etsu Chemical Company; 50.7 g) was added to the EC-10 solution while stirring vigorously until dissolved. TEC (25.4 g) was added to the solution until dissolved/dispersed homogeneously, thereby forming a TPR coating formulation. The IR beads (395 g) prepared in Example 1A were fluid-bed coated with the TPR coating formulation (7.5% solids) in a Glatt 1 equipped with a 4″ Wurster insert at a product temperature of 33±2° C., atomization air pressure of 1.50 bar, inlet air volume of 70-90 m³/h, and a spray flow rate of 3-6 g/min for a TPR coating level of 30% by weight. Samples were pulled at a coating level of 15%, 20% and 25% by weight for drug release testing. Dried beads with a mean particle size of less than about 355 gm were collected by sieving.

### Example 3B

CR Beads: The IR beads (440 g) prepared in Example 1B were fluid-bed coated with EC-10/TEC (ratio: 10/1) solution (5.5% solids) for a coating weight of 20% as previously described in Example 1 C. These SR beads were further coated with a TPR coating formulation (EC-10/HP-55/TEC at 55/30/15; 7.5% solids) in a Glatt 1 for a TPR coating weight of 20% as described in Example 3A, above. Samples were pulled at a coating weight of 10% and 15% for drug release testing. The dried CR beads thus prepared, having a mean particle size of <355 μm were collected by sieving.

FIG. 4 shows the drug release profiles from TPR beads (Example 3A) and the CR beads prepared above in Example 3B, demonstrating the significant effect of the inner barrier layer comprising a water-insoluble polymer on the drug release from coated beads with a mean particle size of less than about 355 μm.

### Example 4A

SR Beads (20% SR Coating): The IR beads (550 g) from Example 1B, above, were fluid-bed coated with an SR functional polymer coating (EC-10/TEC at 10/1) dissolved in acetone/water at 90/10 (5.5% solids) for a coating weight of 20%.

### Example 4B

CR Beads (20% DR Coating on 20% SR Coating): The lot of SR beads from Example 4A, above was further coated with a solution of HP-55/TEC at a ratio of 10/1 (5.5% solids) dissolved in acetone (2278 g)/water (255 g) for a DR (delayed-release) coating weight of 20%. The resulting CR beads were dried in the Glatt for 20 min to drive off residual solvents. The dried beads with a mean particle size of less than about 355 μm were collected by sieving.

### Example 4C

CR Beads (20% DR Coating on 20% TPR Coating): The IR beads (550 g) prepared in Example 1B were first coated with a TPR functional polymer coating (EC-10/HP-55/TEC at 60/25/15) dissolved in a acetone/water at 90/10 (7.5% solids) for a weight gain of 20%. 550 g of TPR beads from this lot were further coated with a solution of HP-55/TEC at a ratio of 10/1 (5.5% solids) dissolved in acetone (2278 g)/water (255 g) for an SR coating weight of 20%. The resulting CR beads were dried in the Glatt for 20 min to drive off residual solvents. The dried beads with a mean particle size of less than about 355 μm were collected by sieving.

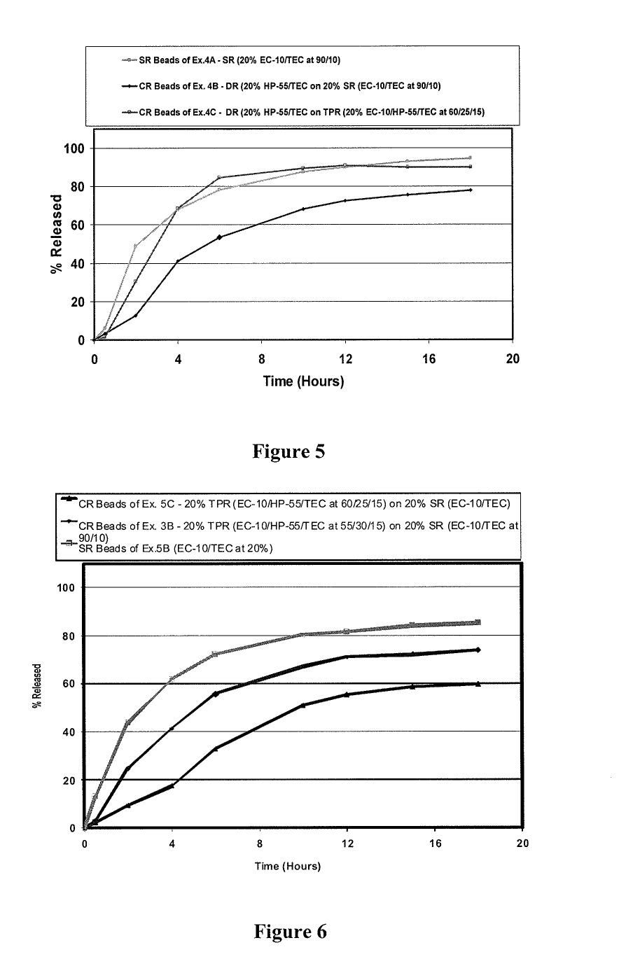

FIG. 5 shows the drug release profiles for SR beads (SR coated IR beads) from Example 4A and CR beads comprising differing dual membrane systems from Examples 4B and 4C.

### Example 5A

IR Beads Comprising MCC Inert Cores: Povidone (PVP K30; 60 g) was slowly added to 75/25 95% ethanol /water (855 g Ethanol 95% and 285 g water) until dissolved under constant stirring for not less than 10 min. Dicyclomine hydrochloride (300 g) was slowly added while stirring until dissolved. The Glatt GPCG 1 equipped with a 6″ bottom spray Wurster 15 cm high column, partition column gap of 15 mm from the ‘B’ bottom air distribution plate covered with a 200 mesh product retention screen (0.8 mm port nozzle) was charged with 1140 g of microcrystalline cellulose spheres (Cellets 100 with a mean particle size of 100 μm from Glatt) and sprayed with the dicyclomine hydrochloride solution (20% solids) at a spray rate of 3-9 g/min, an inlet air volume of 80-100 m3/hr, air atomization pressure of 1.5 bar while maintaining the product temperature of 34±3° C. Following rinsing of the spray system with 50 g of ethanol, the drug-layered beads were dried in the Glatt unit for 30 min to drive off residual solvents (including moisture). The resulting dicyclomine IR beads were sieved through 125 and 250 μm screens to discard oversized particles and fines.

### Example 5B

SR Beads (20% coated with EC-10/TEC): The IR beads (550g) from Example 5A were fluid-bed coated with an SR coating (EC-10/TEC at 10/1) dissolved in a acetone/water at 90/10 mixture (5.5% solids) at a coating weight of 20% as described in Example 2, above.

### Example 5C

CR Beads (30% TPR (60/25/15) on 20% SR (EC-10/TEC): The IR beads (550 g) from Example 5A were first fluid-bed coated with an SR coating (EC-10/TEC at 10/1) dissolved in a acetone/water at 90/10 mixture (5.5% solids) at a coating weight of 20% as described in Example 2, above. This lot of SR beads was further coated with a solution (7.5% solids) of EC-10/HP-55)/TEC at a ratio of 60/25/15 dissolved in 90/10 acetone /water for a TPR coating weight of 30%. The resulting CR beads were dried in the Glatt for 5 min to drive off residual solvents. The dried beads containing about 90% of particles having a mean particle size of less than about 355 μm, and about 99% of particles having a mean particle size of less than about 425 μm, were collected by sieving.

FIG. 6 demonstrates the drug release profiles for SR beads (Example 5B) and CR beads (Example 5C) comprising Cellets 100 (microcrystalline cellulose spheres with an average diameter of 100 μm) in comparison with CR beads (Example 3B) comprising 60-80 mesh sugar spheres (average diameter of 177-250 μm).

### Example 6A

CR Beads (22.5% TPR (60/30/10) on 17.5% SR (EC-10/TEC): The IR beads from Example 5A, above are fluid-bed coated with an SR coating (EC-10/TEC at 10/1) dissolved in a acetone/water at 90/10 mixture (5.5% solids) at a coating weight of 15% as described in Example 2, above. This lot of SR beads is further coated with a solution (7.5% solids) of EC-10/HP-55/TEC at a ratio of 60/30/10 dissolved in 90/10 acetone/water for a TPR coating weight of 20%. The resulting CR beads are dried in the Glatt for 15 min to drive off residual solvents. The dried beads containing particles with a mean particle size of less than about 355 μm are collected by sieving.

### Example 6B

CR Beads (25% DR (90/10) on 17.5% SR (EC-10/TEC): The IR beads from Example 5A are fluid-bed coated with an SR coating (EC-10/TEC at 10/1) dissolved in a acetone/water at 90/10 mixture (5.5% solids) at a coating weight of 17.5% as described in Example 2, above. This lot of SR beads is further coated with a solution (7.5% solids) of HP-55/TEC at a ratio of 90/10 dissolved in 90/10 acetone /water for a DR coating weight of 20% by weight. The resulting CR beads are dried in the Glatt for 15 min to drive off residual solvents. The dried beads containing particles having a mean particle size of less than about 355 μm are collected by sieving.

### Example 6C

Rapidly Dispersing Microgranules: Rapidly dispersing microgranules are prepared following the procedure disclosed in co-pending US Patent Application Publication No. U.S. 2003/0215500, published Nov. 20, 2003, which is hereby incorporated by reference in its entirety for all purposes. Specifically, D-mannitol (152 kg) with an average particle size of approximately 20 um or less (Pearlitol 25 from Roquette, France) is blended with 8 kg of cross-linked povidone (Crospovidone XL-10 from ISP) in a high shear granulator (GMX 600 from Vector), granulated with purified water (approximately 32 kg), wet-milled using a Comil from Quadro, and finally tray-dried to provide microgranules having an LOD (loss on drying) of less than about 0.8%. The dried granules are sieved and oversize material are again milled to produce rapidly dispersing microgranules with an average particle size in the range of approximately 175-300 μm.

### Example 6D

Dicyclomine HCl CR ODTs, 40-mg and 80-mg: Table 1 lists the compositions of orally disintegrating tablets comprising 40-mg or 80-mg dicyclomine HCl as the CR beads of Example 6A. Pharmaceutically acceptable ingredients (i.e., 1 part of a flavor (e.g., peppermint, cherry, or wintergreen), 0.35 part of a sweetener (sucralose), 5 parts of a disintegrant (e.g., crospovidone, sodium starch glycolate, crosslinked sodium carboxymethylcellulose, or low-substituted hydroxypropylcellulose), and 10 parts of microcrystalline cellulose (Avicel PH101 or Ceolus KG-802), are first blended in a V blender to achieve a homogeneously blended pre-mix. 44.59 parts of the rapidly dispersing microgranules (prepared as described in Example 6C, above) are blended with 39.06 parts of the dicyclomine HCl CR beads (Example 6A, above) and the pre-mix previously prepared above, in a twin shell V-blender for sufficient time to obtain a homogeneously blended compression mix. ODTs comprising 40-mg or 80-mg dicyclomine HCl are compressed using a production scale Hata tablet press equipped with an external lubrication system (Matsui Ex-Lube System) under tableting conditions optimized to provide acceptable tableting properties suitable for packaging in HDPE bottles, Aclar 200 blisters with a peel-off paper backing, and/or ‘push-through’ Aclar blister packs. For example, ODTs comprising 40 mg dicyclomine HCl as CR beads are compressed at the following conditions: —tooling: 14 mm round, flat face, radius edge; compression force: 12-16 kN; mean weight: 800 mg; mean hardness: ˜30-60 N; and friability: 0.2-0.4%. Dicyclomine HCl CR ODTs (40 mg or 80 mg) thus produced will rapidly disintegrate in the oral cavity creating a smooth, easy-to-swallow suspension comprising coated dicyclomine HCl beads, having a release profile suitable for a once- or twice-daily dosing regimen. ODT tablets produced with higher amounts of the rapidly dispersing granules will have a marginally better mouthfeel and shorter disintegration time.

TABLE 1
Compositions and Tableting Properties of ODTs

| | Composition (mg/tablet) | | | | | |
|---|---|---|---|---|---|---|
| | Example 6 | | | Example 7 | | |
| Ingredients | %/tablet | 40-mg | 80-mg | %/tablet | 40 mg | 80-mg |
| Dicyclomine CR Beads | 39.06 | 312.5 | 625.0 | 25.80 | 206.4 | 412.8 |
| Rapidly Dispersing | 44.59 | 356.7 | 713.4 | 58.00 | 464.0 | 928.0 |
| Granules | | | | | | |
| MCC - Ceolus or | 10.00 | 80.0 | 160 | 10.00 | 80.0 | 160.0 |
| Avicel | | | | | | |
| Crospovidone (XL-10) | 5.00 | 40.0 | 80.0 | 5.00 | 40.0 | 80.0 |
| Sucralose | 0.35 | 2.8 | 5.6 | 0.35 | 2.8 | 5.6 |
| Peppermint Flavor | 1.0 | 8.0 | 16 | 0.85 | 6.8 | 13.6 |
| Magnesium Stearate | Trace | Trace | Trace | Trace | Trace | Trace |
| Tablet Weight | 100.0% | 800 mg | 1600 mg | 100.0% | 800 mg | 1600 mg |

### Example 7A

Dicyclomine hydrochloride IR Beads (drug load: 30% w/w): Dicyclomine hydrochloride is slowly added with stirring to a solution of a binder (povidone (PVP K30) at 4% by weight) and solvent (95% ethanol and water at a ratio of 75/25) until dissolved as described in Example 1B, above. A Glatt GPCG 3 equipped with a 7″ bottom spray Wurster 8″ high column, “B” bottom air distribution plate covered with a 200 mesh product retention screen, 1.2 mm port nozzle, is charged with 2500 g of Cellets 100 (microcrystalline cellulose spheres), which are then sprayed with the dicyclomine hydrochloride solution (20% solids) at an atomization pressure of 1.8 bar and a spray rate of 10-15 g/min, at an inlet air volume set at 80-125 cubic meter per hour, and an air atomization pressure of 1.8 bar, while maintaining the product temperature of 33±3° C. Following rinsing with 50 g of acetone, a seal-coat solution (Klucel LF dissolved in 85/15 acetone/water, 7.5% solids) is sprayed at an initial rate of 10 g/min, and dried in the Glatt unit for 5 min. to drive off residual solvents (including moisture). The resulting IR beads are sieved to discard oversized (>355 μm or 50 mesh) beads and fines (<100 mesh).

### Example 7B

Dicyclomine CR Beads (20% TPR (60/30/10 on 15% SR (EC-10/TEC): An SR coating solution is prepared by first slowly adding ethylcellulose (EC-10) to a 90/10 acetone/water mixture while stirring until dissolved, followed by the addition of a plasticizer (TEC) as described in Example 2, above. The dicyclomine HCl IR beads from Example 7A, above are charged into a Glatt GPCG 1 equipped with a 6″ bottom spray Wurster 8″ high column, 1.0 mm nozzle port, and a bottom ‘C’ distribution plate, and coated with the SR coating solution at a fluidization air volume of 80-100 m³/hr, atomization air pressure of 1.25 bar, target product temperature of 33° C., and a spray rate of about 6-10 g/min for a weight gain of 15% by weight.

The SR beads from above are sprayed with a TPR coating solution comprising EC-10, HP-55, and TEC at a ratio of 60/30/10 dissolved in a 90/10 acetone/water mixture (7.5% solids) in the same Glatt unit to a coating weight of 20%. Following an acetone rinse, a compressible coating of hydroxypropylcellulose (Klucel LF) dissolved in 85/15 acetone/water, 7.5% solids) is applied as described in Example 7A, above and then dried in the Glatt unit for 15 min. to drive off residual solvents (including moisture).

### Example 7C

Dicyclomine HCl ODT CR, 40-mg and 80-mg: Appropriate quantities of rapidly dispersing microgranules (prepared as described in Example 6C, above) and dicyclomine hydrochloride CR beads from Example 7B are blended with pre-blended pharmaceutically acceptable ingredients (see Table 1, Example 7 for the ingredients and quantities) in a twin shell V-blender for a sufficient time to provide a homogeneously distributed blend suitable for compression. ODTs comprising 40-mg or 80-mg dicyclomine HCl as CR beads are compressed using a production scale Hata Tablet Press equipped with an external lubrication system (Matsui Ex-Lube System) under tabletting conditions optimized to provide acceptable tabletting properties (e.g., typically a friability of not more than 0.5%. Dicyclomine HCl CR ODTs (40 mg or 80 mg) thus produced will rapidly disintegrate in the oral cavity creating a smooth, easy-to-swallow suspension comprising coated dicyclomine HCl CR beads, having a release profile suitable for a once- or twice-daily dosing regimen.

### Example 8A

Tiagabine IR Beads (drug load: 30% w/w): Tiagabine is slowly added with stirring to a solution of a binder (Klucel LF at 4% by weight) until dissolved, similar to the procedure described in Example 1B, above. A Glatt GPCG 3 is charged with 2500 g of Cellets 100 (microcrystalline cellulose spheres), which are then sprayed with the tiagabine solution as described in step 7.A. Following rinsing with 50 g of acetone, a seal-coat solution (Klucel LF dissolved in 85/15 acetone/water, 7.5% solids) is sprayed at an initial rate of 10 g/min, and dried in the Glatt unit for 5 min. to drive off residual solvents (including moisture). The resulting IR beads are sieved to discard oversized (>355 μm or 50 mesh) beads and fines (<100 mesh).

### Example 8B

Tiagabine CR Beads (25% DR (HP-55/TEC at 90/10 on 10% SR (EC-10/TEC): The IR beads from Example 8A, above are charged into a Glatt GPCG 3 and sprayed with an SR coating solution comprising EC-10 and triethylcitrate for a weight gain of 10% by weight.

The SR beads from above are sprayed with a DR coating solution comprising HP-55 and TEC at a ratio of 90/10 dissolved in a 90/10 acetone/water mixture (7.5% solids) in the same Glatt unit to a coating weight of 25%. Following an acetone rinse, a compressible coating of hydroxypropylcellulose (Klucel LF) dissolved in 85/15 acetone/water, 7.5% solids) is applied as described in Example 7A, above and then dried in the Glatt unit for 15 min. to drive off residual solvents (including moisture).

### Example 8C

Tiagabine ODT CR, 20 mg and 40 mg: Appropriate quantities of rapidly dispersing microgranules (prepared as described in Example 6C, above) and tiagabine CR beads from Example 8B are blended with pre-blended pharmaceutically acceptable ingredients in a twin shell V-blender for a sufficient time to provide a homogeneously distributed blend suitable for compression. ODTs comprising 20 mg or 40 mg tiagabine as CR beads are compressed using a production scale Hata Tablet Press equipped with an external lubrication system (Matsui Ex-Lube System) under tabletting conditions optimized to provide acceptable tabletting properties (e.g., typically a friability of not more than 0.5%. Tiagabine CR ODTs (20 mg or 40 mg) thus produced will rapidly disintegrate in the oral cavity creating a smooth, easy-to-swallow suspension comprising coated tiagabine CR beads, having a release profile suitable for a once- or twice-daily dosing regimen.

While the invention has been described in connection with the specific embodiments herein, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

We claim:

1. A controlled release composition comprising a plurality of anti-cholinergic drug-containing particles, the particles comprising:
(a) a core comprising an anti-cholinergic drug;
(b) a first coating disposed over the core comprising at least one water-insoluble polymer; and
(c) a second coating disposed over the core comprising an enteric polymer optionally in combination with a water-insoluble polymer.

2. The controlled release composition of claim 1, wherein the second coating comprises a water-insoluble polymer in combination with an enteric polymer.

3. The controlled release composition of claim 1, wherein the second coating is disposed over the first coating.

4. The controlled release composition of claim 3, wherein the first coating comprises the combination of a water-insoluble polymer and an enteric polymer, and the second coating comprises an enteric polymer.

5. The controlled release composition of claim 2, wherein the second coating is disposed over the first coating.

6. The composition of claim 2, wherein the ratio of the water-insoluble polymer to the enteric polymer is about 10:1 to about 1:1.

7. The controlled release composition of claim 1, wherein at least one of the first and second coatings further comprise a plasticizer.

8. The controlled release composition of claim 5, wherein at least one of said first and second coatings further comprise a plasticizer.

9. The controlled release composition of claim 8, wherein the first and second coatings further comprise a plasticizer.

10. The controlled release composition of claim 1, wherein the core comprises an anti-cholinergic drug coated onto an inert core.

11. The controlled release composition of claim 9, wherein the core comprises an anti-cholinergic drug coated onto an inert core.

12. The composition of claim 1, wherein the core further comprises a polymeric binder selected from the group consisting of hydroxypropyl cellulose, hydroxypropyl methylcellulose, polyvinylpyrrolidone and mixtures thereof.

13. The composition of claim 1 further comprising:
(d) a plurality of rapidly-dispersing microgranules each having an average particle size of not more than about 400 μm and comprising (i) a disintegrant and (ii) a sugar alcohol and/or a saccharide, wherein said sugar alcohol and/or saccharide each have an average particle size of not more than about 30 μm.

14. The composition of claim 13, wherein the ratio of rapidly-dispersing microgranules to anti-cholinergic drug-containing particles ranges from about 6:1 to about 1:2.

15. The composition of claim 13, wherein the rapidly-dispersing microgranules comprise a disintegrant selected from the group consisting of crosslinked polyvinylpyrrolidone, sodium starch glycolate, crosslinked carboxymethylcellulose of sodium, low-substituted hydroxypropylcellulose and mixtures thereof.

16. The composition of claim 2, wherein the second coating comprises about 5 to about 60 wt % relative to the total weight of the anti-cholinergic drug-containing particle.

17. The composition of claim 1, wherein the anti-cholinergic drug is selected from the group consisting of atropine, benactyzine, benztropine, biperiden, butylscopolammonium bromide, cyclopentolate darifenacin, dexetimide, dicyclomine, emepronium, glycopyrrolate, hexahydrosiladifenidol, octylonium, orphenadrine, oxybutynin, oxyphenonium, pirenzepine, procyclidine, propantheline propylbenzilylcholine, quinidine, scopolamine, tolterodine trihexyphenidyl, tropicamide, mivacurium, atracurium, doxacurium, cisatracurium, vecoronium, rocuronium, pancuronium, tabocurarine, gallamine, pipecuronium, hexamethonium, mecamylamine, trimethaphan, succinylcholine, suxamethonium, decamethonium, methoxycoronaridine, mecamylamine, imidafenacin, and pharmaceutically acceptable salts, hydrates, polymorphs, and/or solvates thereof.

18. The composition of claim 1, wherein the anti-cholinergic drug is dicyclomine or salts, polymorphs, and/or solvates thereof

19. The composition of claim 9, wherein the anti-cholinergic drug is dicyclomine or salts and/or hydrates thereof

20. The composition according to claim 1, wherein the water-insoluble polymer is selected from the group consisting of ethylcellulose, cellulose acetate, cellulose acetate butyrate, polyvinyl acetate, neutral methacrylic acid/methylmethacrylate copolymers, and mixtures thereof.

21. The composition of claim 1, wherein the enteric polymer is selected from the group consisting of cellulose acetate phthalate, hydroxypropyl methylcellulose phthalate, hydroxypropyl methylcellulose acetate succinate, polyvinyl acetate phthalate, pH-sensitive methacrylic acid/methylmethacrylate copolymers, shellac, and mixtures thereof

22. The composition of claim 1, wherein the water-insoluble polymer is ethylcellulose and the enteric polymer is hydroxypropylmethylcellulose phthalate.

23. The composition of claim 19, wherein the water-insoluble polymer is ethylcellulose and the enteric polymer is hydroxypropylmethylcellulose phthalate.

24. The composition of claim 1, wherein the anti-cholinergic drug-containing particles exhibit a drug release profile substantially corresponding to the following pattern when dissolution tested using United States Pharmacopoeia Apparatus 2 (paddles @ 50 rpm) in a 2-stage dissolution media (700 mL of 0.1N HCl for the first 2 hrs followed by testing in 900 mL buffer at pH 6.8 obtained by adding 200 mL of a pH modifier) at 37° C.:
after 4 hours, about 40±20% of the total anti-cholinergic drug is released;
after 8 hours, about 65±25% of the total anti-cholinergic drug is released; and
after 12 hours, about 70±30% of the total anti-cholinergic drug is released.

25. A dosage form comprising the controlled release composition of claim 1.

26. A dosage form comprising the controlled release composition of claim 19.

27. The dosage form of claim 26, further comprising:
(d) a plurality of rapidly-dispersing microgranules each having an average particle size of not more than about 400 μm and comprising (i) a disintegrant and (ii) a sugar alcohol and/or a saccharide, wherein said sugar alcohol and/or saccharide each have an average particle size of not more than about 30 μm;
wherein said dosage forth is an orally disintegrating tablet.

28. The dosage form of claim 27, wherein said orally disintegrating tablet substantially disintegrates within about 60 seconds after contact with saliva in the oral cavity or simulated saliva fluid.

29. The dosage form of claim 27, wherein said orally disintegrating tablet substantially disintegrates within about 30 seconds when disintegration is tested according to the USP <701> Disintegration Test.

30. A method of preparing a controlled release composition of claim 1, comprising:
(a) preparing a plurality of cores comprising an anti-cholinergic drug;
(b) coating said cores with said first coating; and
(c) coating said cores with said second coating.

31. The method of claim 30, wherein said coating of step (b) is carried out prior to said coating of step (c).

32. The method of claim 30, further comprising:
(d) granulating a sugar alcohol and/or a saccharide, each having an average particle diameter of not more than about 30 μm, and a disintegrant, thereby producing rapidly disintegrating microgranules with an average particle size not more than about 400 μm;
(e) blending the coated core particles and rapidly disintegrating microgranules;
(f) compressing said blend of coated core particles and rapidly disintegrating microgranules, thereby forming an orally disintegrating tablet.

33. The method of claim 30, wherein the anti-cholinergic drug is selected from the group consisting of atropine, benactyzine, benztropine, biperiden, butylscopolammonium bromide, cyclopentolate darifenacin, dexetimide, dicyclomine, emepronium, glycopyrrolate, hexahydrosiladifenidol, octylonium, orphenadrine, oxybutynin, oxyphenonium, pirenzepine, procyclidine, propantheline propylbenzilylcholine, quinidine, quinuclidinyl benzilate, scopolamine, tolterodine trihexyphenidyl, tropicamide, mivacurium, atracurium, doxacurium, cisatracurium, vecoronium, rocuronium, pancuronium, tabocurarine, gallamine, pipecuronium, hexamethonium, mecamylamine, trimethaphan, succinylcholine, suxamethonium, decamethonium, methoxycoronaridine, mecamylamine, and imidafenacin.

34. The method of claim 33, wherein said anti-cholinergic drug comprises dicyclomine or a salt, polymorph, and/or hydrate thereof

35. The method of claim 32, wherein said orally disintegrating tablet substantially disintegrates within about 60 seconds after contact with saliva in the oral cavity or simulated saliva fluid.

36. The method of claim 32, wherein said orally disintegrating tablet substantially disintegrates within about 30 seconds after contact with saliva in the oral cavity or simulated saliva fluid.

37. A method of treating intestinal hypermotility or irritable bowel syndrome, comprising administering a therapeutic amount of the composition of claim 1 to a patient in need thereof

38. A method of treating intestinal hypermotility or irritable bowel syndrome, comprising administering a therapeutic amount of the dosage form of claim 27 to a patient in need thereof.

39. A method of increasing compliance in a patient suffering from intestinal hypermotility or irritable bowel syndrome, comprising administering a therapeutic amount of the dosage form of claim 27 to a patient in need thereof.

40. A controlled release composition comprising a plurality of drug-containing particles, the particles comprising:
(a) a core comprising a drug;
(b) a first coating disposed over the core comprising at least one water-insoluble polymer; and
(c) a second coating disposed over the core comprising an enteric polymer optionally in combination with a water-insoluble polymer
(d) wherein the drug has a short plasma elimination half-life and requires frequent dosing to minimize adverse events.

41. A controlled release composition a plurality of drug-containing particles, the particles comprising:
(a) a core comprising the drug;
(b) a first coating disposed over the core comprising a water-insoluble polymer; and
(c) a second coating disposed over the first coating comprising an enteric polymer.

42. The controlled release composition of claim 41, wherein the drug is selected from the group consisting of analgesics anticonvulsants, antidiabetic agents, anti-infective agents, anti-Parkinsonian agents, antirheumatic agents, cardiovascular agents, central nervous system (CNS) stimulants, dopamine receptor agonists, anti-emetics, gastrointestinal agents, psychotherapeutic agents, opioid agonists, opioid antagonists, anti-epileptic drugs, histamine H₂ antagonists, anti-asthmatic agents, and skeletal muscle relaxants.
